(19) United States
(12) Patent Application Publication
(10) Pub. No.: US 2025/0041390 A1
(43) Pub. Date: Feb. 6, 2025

(54) COMPOSITIONS AND METHODS FOR TREATING VASCULAR DISEASE

(71) Applicant: CASE WESTERN RESERVE UNIVERSITY, Cleveland, OH (US)

(72) Inventor: Anirban Sen Gupta, Fairview Park, OH (US)

(21) Appl. No.: 18/626,999

(22) Filed: Apr. 4, 2024

Related U.S. Application Data
(60) Provisional application No. 63/494,038, filed on Apr. 4, 2023.

(51) Int. Cl. A61K 38/48; A61K 9/127; A61K 47/42; A61P 7/02

(52) U.S. Cl. CPC A61K 38/484; A61K 9/1271; A61K 47/42; A61P 7/02

(57) ABSTRACT

A composition includes a plurality of clot-targeted thrombin cleavable nanoparticles (CTNPs), each CTNP including a thrombin cleavable shell that defines an outer surface of the CTNP, a core, which is loaded with plasmin, and a plurality of platelet binding peptides (PBPs) and fibrin binding peptides (FBPs) that are linked to the shell and extend from the outer surface, wherein the CTNP is configured to adhere to clots, activated platelets, and/or fibrin upon systemic administration to a subject, shield the loaded plasmin in circulation from neutralization, and release the loaded plasmin at the clot site by thrombin triggered degradation of the CTNP.

### CROSS-REFERENCE TO RELATED APPLICATIONS

This application claims priority from U.S. Provisional Application No. 63/494,038, filed Apr. 4, 2023, the subject matter of which is incorporated herein by reference in its entirety.

### SEQUENCE LISTING

The instant application contains a Sequence Listing which has been submitted electronically in XML format and is hereby incorporated by reference in its entirety. Said XML copy, created on Apr. 2, 2024, is named CWR-032414US ORD.st.26 and is 3,754 bytes in size.

### BACKGROUND

Thrombo-occlusive vascular diseases, such as atherosclerosis, myocardial infraction, stroke, deep vein thrombosis, and pulmonary embolism, continue to be the leading causes of morbidity and mortality in the world. Beyond the already existent global burden of such pathologies, the emergence, and sustained effects of the COVID-19 pandemic has given rise to new challenges of thrombosis and thromboinflammation in the acute as well as chronic phases of patients with COVID-19. Irrespective of etiology, one common end-point of such thrombotic pathologies is the formation of insoluble crosslinked protein fibrin as a major component of the occlusive blood clot. Therefore, traditional and emerging therapeutic approaches remain focused on the utilization of drugs that can reduce fibrin formation (e.g., anticoagulants) and that can degrade already formed fibrin (e.g., fibrinolytics).

Current clinical approaches for fibrinolytic therapy use tissue plasminogen activators (alteplase, reteplase, etc.), which work by converting plasminogen to plasmin that can then break down fibrin. Although the plasminogen to plasmin conversion by such drugs is significantly enhanced for fibrin-associated plasminogen, these drugs are administered systemically (oral or intravenous) in a highly regulated regimen and activate circulating plasminogen leading to systemic fibrinogenolysis, resulting in significant coagulopathy and hemorrhagic risks.

### SUMMARY

Embodiments described herein relate to clot-targeted thrombin-cleavable nanoparticles (CTNPs) and to their use in delivering plasmin to fibrin-rich clots and treating thrombo-occlusive pathologies in a subject in need thereof. We constructed a nanomedicine platform that can actively target to clot site and deliver an encapsulated plasmin payload as a direct fibrinolytic agent in a thrombin-responsive manner. Our in vitro studies establish the reproducibility of manufacture and the mechanism of action of this plasmin-loaded CTNP system. Our in vitro and in vivo studies demonstrate that encapsulation within CTNPs can provide a way to protect plasmin from rapid neutralization in circulation and then to deliver this plasmin specifically to a clot for thrombin-triggered release to render localized fibrinolysis. The CTNPs described herein can provide an efficacious way of directly using plasmin for fibrinolytic therapies, and potentially avoiding the systemic side-effects of current fibrinolytic approaches involving plasminogen activators.

In some embodiments, each CTNP can include a thrombin cleavable shell that defines an outer surface of the CTNP, a core, which is loaded with plasmin, and a plurality of platelet binding peptides (PBPs) and fibrin binding peptides (FBPs) that are linked to the shell and extend from the outer surface. The CTNP is configured to adhere to clots, activated platelets, and/or fibrin upon systemic administration to a subject, shield the loaded plasmin in circulation from neutralization, and release the loaded plasmin at the clot site by thrombin triggered degradation of the CTNP.

In some embodiments, the CTNPs can bind to the clot under a hemodynamic shear environment.

In some embodiments, a composition including the CTNPs can provide localized thrombolytic and/or fibrinolytic action at the clot site.

In some embodiments, the shell of the CTNP can include at least one phospholipid and a thrombin cleavable lipopeptide conjugate that triggers degradation of the CTNP and release of the plasmin at the clot site.

In some embodiments, the CTNP can have a diameter of about 50 nm to about 5 m, preferably about 50 nm to about 250 nm, or more preferably about 150 nm to about 200 nm.

In some embodiments, the CTNP is a liposome. The liposome can include a plurality of phospholipids and optionally cholesterol to define a lipid membrane. The phospholipids can include ate least one of distearoylphosphatidylserine (DSPS), distearoylphosphatidylcholine dipalmitoylphosphatidylcholine (DSPC), dihexadecanoylglycerophosphoethanolamine (DHPE), dibehenoylglycerophosphocoline (DBPC), distearoylphosphatidylcholine (DSPC), diarachidonylphosphatidylcholine (DAPC), dioleoylphosphatidylethanolamine (DOPE), dipalmitoylphosphatidylethanolamine (DPPE), and distearoylphosphatidylethanolamine (DSPE); dipalmitoylphosphatidic acid (DPPA), or PEG functionalized lipids thereof.

In some embodiments, the PBPs and FBPs are conjugated to the phospholipids with PEG linkers.

In some embodiments, the PBP and FBP conjugated phospholipids can include about 1 mole % to about 10 mole %, preferably about 2.5 mole % to about 10 mole % of the total lipid composition of the liposome.

In some embodiments, the liposome can include DSPE conjugated to PBP with PEG (DSPE-PEG-PBP), DSPE conjugated to FBP with PEG (DSPE-PEG-FBP), a thrombin-cleavable lipopeptide conjugate, and cholesterol.

In some embodiments, the PBPs and FBPs are spatially or topographically arranged on the outer surface such that the PBPs and FBPs do not spatially mask each other and the nanoparticle is able to adhere to a clot site with exposed activated surface integrin αIIβ3 and fibrin.

In some embodiments, the CTNPs can have a shape, size and elastic modulus that facilitates margination to a clot site upon administration to vasculature of a subject.

In some embodiments, the PBPs can have an amino acid sequence of SEQ ID NO: 1 (CGSSSGRGDSPA) and the FBPs have an amino acid sequence of SEQ ID NO: 2 (cyclo-AC-Y(DGI)C(HPr)YGLCYIQGK-Am).

In some embodiments, the ratio of PPB:FPB: is about 25:75 to about 75:25.

In some embodiments, the thrombin cleavable lipopeptide conjugate includes a thrombin cleavable peptide. The thrombin cleavable peptide can have an amino acid sequence of SEQ ID NO: 3(DVTPRC).

In some embodiments, the amount of plasmin delivered to a clot site in a subject by the CTNPs is an amount effective to promote thrombolysis and/or fibrinolysis of the clot in the subject.

Other embodiments described herein relate to a method of delivering plasmin to a clot site in a subject. The method includes administering to the subject a composition comprising a plurality of CTNPs described herein.

Still other embodiments described herein relate to method of treating a clot in vasculature of a subject in need thereof. The method includes administering to the subject a composition comprising a plurality of CTNPs described herein.

### BRIEF DESCRIPTION OF DRAWINGS

FIG. 1 illustrates design and mechanism of action of plasmin-loaded clot-targeted thrombin-cleavable nanoparticles (CTNPs) for targeted fibrinolysis; CTNPs anchor to clots via platelet-binding peptides (PBP binding to αIIbβ3 on activated platelets) and fibrin-binding peptides (FBP), and undergo thrombin-triggered destabilization for site-localized release of plasmin for fibrinolysis.

FIGS. 2(A-C) illustrate: (A) Representative dynamic light scattering analysis and cryo-TEM imaging data for CTNPs show that pre-extrusion the multilamellar vesicle diameter is ˜1 μm and upon extrusion the unilamellar vesicle diameter is ˜150 nm (scale bar in cryo-TEM images is 200 nm); (B) Plasmin encapsulation analysis shows that plasmin can be reproducibly loaded at ˜350-500 nM per batch of CTNPs; (C) Release kinetics analysis shows that over a 2-hour period the diffusive release of plasmin from CTNPs is low (˜40% release by 120 min) whereas thrombin-triggered release of plasmin from CTNPs is significantly high (˜40% by 30 min and ˜70% by 120 min).

FIG. 3 illustrates representative fluorescent images and quantitative data showing that compared to control nanoparticles (no peptide decoration), CTNPs can significantly anchor onto clots under flow. Scale bar in fluorescence images: 10 μm; **** p≤0.0001.

FIG. 4 illustrates D-dimer ELISA analysis using a well-plate based assay shows that plasmin-loaded CTNPs can render modest levels of fibrinolysis over 30 min when plasmin is diffusively released and this fibrinolytic effect is significantly increased when release of plasmin is enhanced by exogenously added thrombin trigger; Treatment with free plasmin added directly was used as positive control and treatment with saline was used as negative control. * p≤0.05, ** p≤0.01.

FIGS. 5(A-C) illustrate: (A) Representative fluorescence images of clots under flow of various treatment groups in the BioFlux microfluidic channels show that plasmin-loaded CTNPs in the presence of exogenously added thrombin can rapidly lyse clots at a level similar to free plasmin effect; (B) Kinetic analysis of ‘fibrin fluorescence loss’ confirms that plasmin-loaded CTNPs in the presence of exogenously added thrombin rapidly lyse clots at a level similar to free plasmin effect, while plasmin in the presence of exogenously added antiplasmin or plasmin-loaded CTNPs in the absence of exogenously added thrombin render minimal fibrinolysis; (C) D-dimer analysis of lysis products from the outlet well of BioFlux experiments after the 30-min time-point confirm the enhanced fibrinolytic effect of plasmin-loaded CTNPs in presence of exogenously added thrombin. * p≤0.05, ** p≤0.01, *** p≤0.001.

FIGS. 6(A-C) illustrate (A) experimental setup for plasmin-loaded CTNP evaluation in the zebrafish venous thrombosis model, with representative brightfield images showing clot formation post laser-injury; (B) Time-to-occlusion study results showing that without treatment or with ‘control nanoparticle’ treatment the vessel occluded rapidly (<25 sec) and this was also observed for ‘free plasmin’ treatment likely because plasmin is rapidly inhibited upon administration thereby preventing any therapeutic action; in contrast, treatment with plasmin-loaded CTNPs was able to significantly prevent clot formation such that vessel occlusion was not observed up to 120 sec; (C) Time-to-recanalization study results showing that without treatment or with ‘control nanoparticle’ treatment no recanalization was observed for 30 min, while with plasmin-loaded CTNP treatment the vessel was recanalized in <20 min due to effective fibrinolysis. *** p≤0.001**** p≤0.0001.

FIG. 7 illustrates chemical structures and mass spectrometric characterization of DSPE-PEG2000-PBP.

FIG. 8 illustrates chemical structures and mass spectrometric characterization of DSPE-PEG2000-FBP.

FIG. 9 illustrates chemical structures and mass spectrometric characterization of Stearyl-TCP conjugation and its thrombin-induced degradation.

FIG. 10 illustrates a schematic depicting the thin film rehydration followed by extrusion process in the manufacturing of CTNPs.

FIG. 11 illustrates a schematic depicting the parallel plate flow chamber (PPFC) setup to study the clot-targeting efficacy of CTNPs.

FIG. 12 illustrates a schematic depicting the BioFlux microfluidic setup to study the fibrinolytic effect of plasmin-loaded CTNPs under vascularly relevant shear flow condition.

FIG. 13 illustrates fibrin fluorescence intensity loss under various treatments in BioFlux microfluidics.

FIGS. 14(A-C) illustrate (A) Representative fibrinolysis kinetics data from BioFlux studies comparing treatment of clots with free plasmin (positive control), ‘plasmin+antiplasmin’ (negative control), plasmin-loaded CTNPs with vs. without thrombin trigger, and plasmin-loaded CNPs with thrombin trigger; (B) D-dimer ELISA based comparison of fibrin degradation products in outlet well fluid in BioFlux studies comparing treatment of clots with plasmin-loaded CTNPs with vs. without thrombin trigger and plasmin-loaded CNPs with thrombin trigger; (C) Time-to-occlusion (TTO) and Time-to-recanalization (TTR) studies in the zebrafish model comparing plasmin-loaded CTNPs vs. CNPs.

### DETAILED DESCRIPTION OF THE INVENTION

All scientific and technical terms used in this application have meanings commonly used in the art unless otherwise specified. The definitions provided herein are to facilitate understanding of certain terms used frequently herein and are not meant to limit the scope of the application.

The articles “a” and “an” are used herein to refer to one or to more than one (i.e., to at least one) of the grammatical object of the article. By way of example, “an element” means one element or more than one element.

The term “about” will be understood by persons of ordinary skill in the art and will vary to some extent on the context in which it is used.

The verb “comprise” as is used in this description and in the claims and its conjugations are used in its non-limiting sense to mean that items following the word are included, but items not specifically mentioned are not excluded. The present invention may suitably “comprise”, “consist of”, or “consist essentially of”, the steps, elements, and/or reagents described in the claims.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

The term “pharmaceutically acceptable” means suitable for use in contact with the tissues of humans and animals without undue toxicity, irritation, allergic response, and the like, commensurate with a reasonable benefit/risk ratio, and effective for their intended use within the scope of sound medical judgment.

The term “subject” or “animal” can refer to any animal including, but not limited to, humans and non-human animals (e.g., rodents, arthropods, insects, fish (e.g., zebrafish)), non-human primates, ovines, bovines, ruminants, lagomorphs, porcines, caprines, equines, or canines felines, aves, etc.).

The terms “diminishing,” “reducing,” or “preventing,” “inhibiting,” and variations of these terms, as used herein include any measurable decrease, including complete or substantially complete inhibition. The terms “enhance” or “enhanced” as used herein include any measurable increase or intensification.

As used herein, the term “polypeptide” refers to a polymer composed of amino acid residues related naturally occurring structural variants, and synthetic non-naturally occurring analogs thereof linked via peptide bonds or modified peptide bonds (i.e., peptide isosteres), related naturally occurring structural variants, and synthetic non-naturally occurring analogs thereof, glycosylated polypeptides, and all “mimetic” and “peptidomimetic” polypeptide forms. Synthetic polypeptides can be synthesized, for example, using an automated polypeptide synthesizer. The term can refer to an oligopeptide, peptide, polypeptide, or protein sequence, or to a fragment, portion, or subunit of any of these. The term “protein” typically refers to large polypeptides. The term “peptide” typically refers to short polypeptides.

Conventional notation is used herein to portray polypeptide sequences: the left-hand end of a polypeptide sequence is the amino-terminus; the right-hand end of a polypeptide sequence is the carboxyl-terminus.

An “effective amount” can refer to that amount of a therapeutic agent that results in amelioration of symptoms or a prolongation of survival in the subject and relieves, to some extent, one or more symptoms of the disease or returns to normal (either partially or completely) one or more physiological or biochemical parameters associated with or causative of the disease. “Therapeutic agents” can include any agent (e.g., molecule, drug, pharmaceutical composition, etc.) capable of be encapsulated by or conjugated to a nanoparticle or microparticle construct of the application and further capable of preventing, inhibiting, or arresting the symptoms and/or progression of a disease.

“Nanoparticle” or “microparticle” as used herein is meant to include particles, spheres, capsules, and other structures having a length or diameter of about 10 nm to about 100 μm. For the purposes of this application, the terms “nanosphere”, “nanoparticle”, “nanoparticle construct”, “nanovehicle”, “nanocapsule”, “microsphere”, “microparticle”, and “microcapsule” are used interchangeably.

The phrase “therapeutically effective amount” or “pharmaceutically effective amount” is an art-recognized term. In certain embodiments, the term refers to an amount of a therapeutic agent that produces some desired effect at a reasonable benefit/risk ratio applicable to any medical treatment. In certain embodiments, the term refers to that amount necessary or sufficient to eliminate, reduce or maintain a target of a particular therapeutic regimen. The effective amount may vary depending on such factors as the disease or condition being treated, the particular targeted constructs being administered, the size of the subject or the severity of the disease or condition. One of ordinary skill in the art may empirically determine the effective amount of a particular compound without necessitating undue experimentation. In certain embodiments, a therapeutically effective amount of a therapeutic agent for in vivo use will likely depend on a number of factors, including: the rate of release of an agent from a polymer matrix, which will depend in part on the chemical and physical characteristics of the polymer; the identity of the agent; the mode and method of administration; and any other materials incorporated in the polymer matrix in addition to the agent.

“Optional” or “optionally” means that the subsequently described circumstance may or may not occur, so that the description includes instances where the circumstance occurs and instances where it does not.

Throughout the description, where compositions are described as having, including, or comprising, specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the compositions and methods described herein remains operable. Moreover, two or more steps or actions can be conducted simultaneously.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual and partial numbers within that range, for example, 1, 2, 3, 4, 5, 5.5 and 6. This applies regardless of the breadth of the range.

Embodiments described herein relate to clot-targeted thrombin-cleavable nanoparticles (CTNPs) and to their use in delivering plasmin to fibrin-rich clots and treating thrombo-occlusive pathologies, such as preventing clot occlusion and promoting clot dissolution (accelerating recanalization), in a subject in need thereof. The CTNPs can actively target a clot site and deliver an encapsulated plasmin payload (thus protected from rapid neutralization in circulation) as a direct fibrinolytic agent in a thrombin-responsive manner. The CTNPs are capable of (1) specifically binding to activated platelets and fibrin (for clot-anchorage), and (2) then destabilizing under the action of thrombin (upregulated at thrombus site) for clot-localized payload release.

For plasmin-loaded CTNP construction, we utilized hetero-multivalent surface-decoration of nanoparticles, such as liposomes, with 2 peptides, one binding to activated platelet integrin αIIbβ3 and the other binding to fibrin. Furthermore, we developed a novel lipopeptide by conjugating a thrombin cleavable peptide (TCP) to stearylamine (thus forming TCP-stearate), and this was combined with the clot-targeting lipid-peptide components for the final CTNP construction. The stearyl-amine has an equivalent length of hydrocarbon tail as two other major lipids (e.g., DSPC and DSPE) of our liposomal construct and thus it can assemble efficiently within the liposomal membrane with the hydrophilic TCP motif remaining close to the particle surface for accessibility by thrombin. As the CTNPs bind to the clot site via PBP- and FBP-mediated anchorage, the thrombin can cleave the TCP motif off TCP-stearate, rendering the nanoparticle membrane unstable (due to loss of amphiphilicity) and thus release the payload (plasmin) locally.

We have demonstrated that plasmin-loaded CTNPs can provide diffusive release of plasmin from the CTNPs from a time after administration of at least two hours, whereas thrombin-triggered destabilization of CTNPs significantly enhances this release. Our in vitro microfluidic studies demonstrated that CTNPs can actively anchor onto “platelets+fibrin”-rich clots under shear flow, and the thrombin-triggered plasmin release from CTNPs can enhance the “targeted fibrinolysis” capability. Therapeutic studies in a zebrafish model further demonstrated that the fibrinolytic capability of the CTNP-delivered plasmin is maintained in vivo for effective clot prevention (preventing occlusion) and clot dissolution (accelerating recanalization).

In some embodiments, the CTNPs can each include a thrombin cleavable shell that defines an outer surface of the CTNP and a core, which is loaded with plasmin. A plurality of platelet binding peptides (PBPs) and fibrin binding peptides (FBPs) can be linked to the shell and extend from the outer surface. The CTNPs are configured to adhere to clots, activated platelets, and/or fibrin upon systemic administration to a subject, shield the loaded plasmin in circulation from neutralization, and release the loaded plasmin at the clot site by thrombin triggered degradation of the nanoparticle.

The CTNPs can be made from any biocompatible, biodegradable material that can form a flexible CTNP to which the peptides described herein can be attached, conjugated, and/or decorated and which can be loaded with plasmin prior to administration to a subject, shield the plasmin in circulation to protect plasmin from rapid neutralization in circulation and then to deliver this plasmin specifically to a clot for thrombin-triggered release to render localized fibrinolysis. In some embodiments, the biocompatible, biodegradable flexible nanoparticles can include a liposome, lipidic nanoparticles, dendrimers, a hydrogel, micelle, polymer, and/or a combination of these materials that can include and/or be surface modified or engineered with the PBPs and FBPs, loaded with plasmin, and release the plasmin by diffusion from the nanoparticle and/or thrombin triggered degradation of the nanoparticle.

In some embodiments, the CTNPs can have a shape, size and elastic modulus that facilitates margination to a clot site upon administration to vasculature of a subject. For example, the nanoparticles can have an average or median diameter of about 50 nm to about 5 m, preferably about 50 nm to about 250 nm, or more preferably about 150 nm to about 200 nm. In general, the CTNPs can have dimensions small enough to allow the CTNPs to be systemically administered to a subject and targeted to clots in the vasculature of the subject. In some embodiments, the CTNPs can have a size that facilitates encapsulation of plasmin and optionally one or more therapeutic and/or imaging agents.

In some embodiments, the CTNPs bind to fibrin-rich clot and/or activated platelet site under a hemodynamic shear environment, preferably, under flow of about 5 to about 60 dynes/cm².

The CTNPs may be uniform (e.g., being about the same size) or of variable size. Particles may be any shape (e.g., spherical or rod shaped), but are preferably made of regularly shaped material (e.g., spherical). Other geometries can include substantially spherical, circular, triangle, quasi-triangle, square, rectangular, hexagonal, oval, elliptical, rectangular with semi-circles or triangles and the like.

In some embodiments, the CTNPs can include lipidic nanoparticles, polymer nanoparticles, liposomes, and/or dendrimers with a membrane, shell, or surface. The lipidic nanoparticles, polymer nanoparticles, liposomes, and/or dendrimers can be formed from naturally-occurring, synthetic or semi-synthetic (i.e., modified natural) materials that can be loaded with plasmin, and release the plasmin by diffusion from the CTNPs and/or thrombin triggered degradation of the CTNP.

In some embodiments, the lipidic CTNPs or liposomes can include a membrane or shell that is formed from a naturally-occurring, synthetic or semi-synthetic material that is generally amphipathic (i.e., including a hydrophilic component and a hydrophobic component). Examples of materials that can be used to form the membrane or shell of the lipidic nanoparticle or liposome include lipids, such as fatty acids, neutral fats, phospholipids, oils, glycolipids, surfactants, cholesterol, aliphatic alcohols, waxes, terpenes and steroids, as well as semi-synthetic or modified natural lipids. Semi-synthetic or modified natural lipids can include natural lipids that have been chemically modified in some fashion. The lipid can be neutrally-charged, negatively-charged (i.e., anionic), or positively-charged (i.e., cationic). Examples of anionic lipids can include phosphatidic acid, phosphatidyl glycerol, and fatty acid esters thereof, amides of phosphatidyl ethanolamine, such as anandamides and methanandamides, phosphatidyl inositol and fatty acid esters thereof, cardiolipin, phosphatidyl ethylene glycol, acidic lysolipids, sulfolipids and sulfatides, free fatty acids, both saturated and unsaturated, and negatively-charged derivatives thereof. Examples of cationic lipids can include N-[1-(2,3-dioleoyloxy)propyl]-N,N,N-trimethyl-ammonium chloride and common natural lipids derivatized to contain one or more basic functional groups.

Other examples of lipids, any one or combination of which may be used to form the membrane or shell of the lipidic nanoparticle or liposome can include: phosphocholines, such as 1-alkyl-2-acetoyl-sn-glycero 3-phosphocholines, and 1-alkyl-2-hydroxy-sn-glycero 3-phosphocholines; phosphatidylcholine with both saturated and unsaturated lipids, including dioleoylphosphatidylcholine, dimyristoylphosphatidylcholine, dipentadecanoylphosphatidylcholine, dilauroylphosphatidylcholine, distearoylphosphatidylserine (DSPS), dipalmitoylphosphatidylcholine (DPPC), distearoylphosphatidylcholine (DSPC), and diarachidonylphosphatidylcholine (DAPC); phosphatidylethanolamines, such as dioleoylphosphatidylethanolamine, dipalmitoylphosphatidylethanolamine (DPPE), and distearoylphosphatidylethanolamine (DSPE); phosphatidylglycerols, including distearoylphosphatidylglycerol (DSPG); phosphatidylinositol; sphingolipids, such as sphingomyelin; glycolipids, such as ganglioside GM1 and GM2; glucolipids; sulfatides; glycosphingolipids; phosphatidic acids, such as dipalmitoylphosphatidic acid (DPPA) and distearoylphosphatidic acid (DSPA); palmitic acid; stearic acid; arachidonic acid; oleic acid; lipids bearing polymers, such as chitin, hyaluronic acid, polyvinylpyrrolidone or polyethylene glycol (PEG); lipids bearing sulfonated mono-, di-, oligo- or polysaccharides; cholesterol, cholesterol sulfate, and cholesterol hemisuccinate; tocopherol hemisuccinate; lipids with ether and ester-linked fatty acids; polymerized lipids (a wide variety of which are well known in the art); diacetyl phosphate; diacetyl phosphate; stearylamine; cardiolipin; phospholipids with short chain fatty acids of about 6 to about 8 carbons in length; synthetic phospholipids with asymmetric acyl chains, such as one acyl chain of about 6 carbons and another acyl chain of about 12 carbons; ceramides; non-ionic liposomes including niosomes, such as polyoxyalkylene (e.g., polyoxyethylene) fatty acid esters, polyoxyalkylene (e.g., polyoxyethylene) fatty alcohols, polyoxyalkylene (e.g., polyoxyethylene) fatty alcohol ethers, polyoxyalkylene (e.g., polyoxyethylene) sorbitan fatty acid esters (e.g., the class of compounds referred to as TWEEN (commercially available from ICI Americas, Inc., Wilmington, DE), glycerol polyethylene glycol oxystearate, glycerol polyethylene glycol ricinoleate, alkyloxylated (e.g., ethoxylated) soybean sterols, alkyloxylated (e.g., ethoxylated) castor oil, polyoxyethylene-polyoxypropylene polymers, and polyoxyalkylene (e.g., polyoxyethylene) fatty acid stearates; sterol aliphatic acid esters including cholesterol sulfate, cholesterol butyrate, cholesterol isobutyrate, cholesterol palmitate, cholesterol stearate, lanosterol acetate, ergosterol palmitate, and phytosterol n-butyrate; sterol esters of sugar acids including cholesterol glucuronide, lanosterol glucuronide, 7-dehydrocholesterol glucuronide, ergosterol glucuronide, cholesterol gluconate, lanosterol gluconate, and ergosterol gluconate; esters of sugar acids and alcohols including lauryl glucuronide, stearoyl glucuronide, myristoyl glucuronide, lauryl gluconate, myristoyl gluconate, and stearoyl gluconate; esters of sugars and aliphatic acids including sucrose laurate, fructose laurate, sucrose palmitate, sucrose stearate, glucuronic acid, gluconic acid and polyuronic acid; saponins including sarsasapogenin, smilagenin, hederagenin, oleanolic acid, and digitoxigenin; glycerol dilaurate, glycerol trilaurate, glycerol dipalmitate, glycerol and glycerol esters including glycerol tripalmitate, glycerol distearate, glycerol tristearate, glycerol dimyristate, glycerol trimyristate; long chain alcohols including n-decyl alcohol, lauryl alcohol, myristyl alcohol, cetyl alcohol, and n-octadecyl alcohol; 6-(5-cholesten-3β-yloxy)-1-thio-β-D-galactopyranoside; digalactosyldiglyceride; 6-(5-cholesten-3β-yloxy)hexyl-6-amino-6-deoxy-1-thio-β-D-galactopyranoside; 6-(5-cholesten-3β-yloxy)hexyl-6-amino-6-deoxyl-1-thio-α-D-mannopyranoside; 12-(((7′-diethylaminocoumarin-3-yl)carbonyl)methylamino)octadecanoic acid; N-[12-(((7′-diethylaminocoumarin-3-yl)carbonyl)methylamino)octadecanoyl]-2-aminopalmitic acid; cholesteryl(4′-trimethylammonio)butanoate; N-succinyldioleoylphosphatidylethanolamine; 1,2-dioleoyl-sn-glycerol; 1,2-dipalmitoyl-sn-3-succinylglycerol; 1,3-dipalmitoyl-2-succinylglycerol; 1-hexadecyl-2-palmitoylglycerophosphoethanolamine and palmitoylhomocysteine; and/or any combinations thereof.

Examples of biocompatible, biodegradable polymers that can be used to form the nanoparticles are poly(lactide)s, poly(glycolide)s, poly(lactide-co-glycolide)s, poly(lactic acid)s, poly(glycolic acid)s, poly(lactic acid-co-glycolic acid)s, polycaprolactone, polycarbonates, polyesteramides, polyanhydrides, poly(amino acids), polyorthoesters, polyacetyls, polycyanoacrylates, polyetheresters, poly(dioxanone)s, poly(alkylene alkylate)s, copolymers of polyethylene glycol and poly(lactide)s or poly(lactide-co-glycolide)s, biodegradable polyurethanes, and blends and/or copolymers thereof.

Other examples of materials that may be used to form the CTNPs can include chitosan, poly(ethylene oxide), poly(lactic acid), poly(acrylic acid), poly(vinyl alcohol), poly(urethane), poly(N-isopropyl acrylamide), poly(vinyl pyrrolidone) (PVP), poly(methacrylic acid), poly(p-styrene carboxylic acid), poly(p-styrenesulfonic acid), poly(vinylsulfonicacid), poly(ethyleneimine), poly(vinylamine), poly(anhydride), poly(L-lysine), poly(L-glutamic acid), poly(gamma-glutamic acid), poly(carprolactone), polylactide, poly(ethylene), poly(propylene), poly(glycolide), poly(lactide-co-glycolide), poly(amide), poly(hydroxylacid), poly(sulfone), poly(amine), poly(saccharide), poly(HEMA), poly(anhydride), gelatin, glycosaminoglycans (GAG), poly(hyaluronic acid), poly(sodium alginate), alginate, albumin, hyaluronan, agarose, polyhydroxybutyrate (PHB), copolymers thereof, and blends thereof.

In some embodiments, the liposomes can include a plurality of phospholipids and optionally cholesterol to define the lipid membrane. The phospholipids can include at least one of distearoylphosphatidylserine (DSPS), distearoylphosphatidylcholine dipalmitoylphosphatidylcholine (DSPC), dihexadecanoylglycerophosphoethanolamine (DHPE), dibehenoylglycerophosphocoline (DBPC), distearoylphosphatidylcholine (DSPC), diarachidonylphosphatidylcholine (DAPC), dioleoylphosphatidylethanolamine (DOPE), dipalmitoylphosphatidylethanolamine (DPPE), and distearoylphosphatidylethanolamine (DSPE); dipalmitoylphosphatidic acid (DPPA), or PEG functionalized lipids thereof.

In some embodiments, the lipid membrane can include at least one thrombin cleavable lipopeptide that is amenable to degradation by thrombin. For example, a thrombin cleavable peptide (TCP), such as a recombinant TCP having the amino acid sequence of DVTPRC (SEQ ID NO: 3) can be conjugated to stearylamine (thus forming TCP-stearate), and combined with the clot-targeting lipid-peptide components for the final CTNP construction. The stearyl-amine has an equivalent length of hydrocarbon tail as two other major lipids (e.g., DSPC and DSPE) of the shell of the CTNP and thus it can assemble efficiently within the liposomal membrane with the hydrophilic TCP motif remaining close to the particle surface for accessibility by thrombin. As the CTNPs bind to the clot site via PBP- and FBP-mediated anchorage, the thrombin can cleave the TCP motif off TCP-stearate, rendering the nanoparticle membrane unstable (due to loss of amphiphilicity) and thus release the payload (plasmin) locally. The TCP stearate can be provided in the lipid membrane at about 10 mole % to about 50 mole %, about 15 mole % to about 45 mole %, or about 20 mole % to about 40 mole % of the lipid membrane.

In certain embodiments, the PBPs and FBPs specifically bind to respectively, exposed, activated platelets and fibrin at a clot site. The PBPs and FBPs specifically bind to an activated platelets and fibrin if they bind to or associate with the activated platelets and fibrin with an affinity or Ka (that is, an equilibrium association constant of a particular binding interaction with units of 1/M) of, for example, greater than or equal to about 10¹ M⁻¹. In certain embodiments, the PBPs and FBPs bind to the activated platelets and fibrin at a clot site with a Ka greater than or equal to about 10⁶ M⁻¹, 10⁷ M⁻¹, 10⁸ M⁻¹, 10⁹ M⁻¹, 10¹⁰ M⁻¹, 10¹¹ M⁻¹, 10¹² M⁻¹, or 10¹³ M⁻¹. Alternatively, affinity may be defined as an equilibrium dissociation constant (KD) of a particular binding interaction with units of M (e.g., 10⁻⁵ M to 10⁻¹³ M, or less). In certain aspects, specific binding means binding to the activated platelets and fibrin at the clot site with a KD of less than or equal to about 10⁻⁵ M, less than or equal to about 10⁻⁶ M, less than or equal to about 10⁻⁷ M, less than or equal to about 10⁻⁸ M, or less than or equal to about 10⁻⁹ M, 10⁻¹⁰ M, 10⁻¹¹ M, or 10⁻¹² M or less.

In some embodiments, the PBPs and FBPs are spatially or topographically arranged on the outer surface such that the PBPs and FBPs do not spatially mask each other and the CTNP is able to adhere to a clot site.

In some embodiments, the PBP for activated platelet binding can include a recombinant PBP that binds to the activated platelet surface integrin αIIbβ3. The activated platelet surface integrin αIIbβ3 binding PBPs can have an amino acid sequence of SEQ ID NO: 1 (CGSSSGRGDSPA). The FBP can have an amino acid sequence of SEQ ID NO: 2 (cyclo-AC-Y(DGI)C(HPr)YGLCYIQGK-Am). Peptides having an amino acid sequence of SEQ ID NO: 1 or SEQ ID NO: 2 can be synthesized using fluorenylmethyloxycarbonyl chloride (FMoc)-based solid phase chemistry on Knorr resin, and characterized using mass spectroscopy.

Advantageously, the PBPs and FBPs can each include about 5 to about 30 amino acids. By limiting the size of the peptides to about 5 to about 30 amino acids, the PBPs and FBPs can be spatially or topographically arranged on the CTNP outer surface such that the PBPs and FBPs do not spatially mask each other and are able to adhere to a clot site with exposed activated platelet surface integrin αIIbβ3 and fibrin and promote arrest and optionally aggregation of clots.

The ratio of PPBs to FPBs provided on the CTNP outer surface can be about 75:25 to about 25:75 and be adjusted accordingly to maximize adhesion under low-to-high shear conditions.

Optionally, the nanoparticles can further include a plurality of fibrinogen mimetic peptides (FMPs) that bind to GPIIb-IIIa, endothelial cell targeting peptides, and/or other platelet targeting peptides that are linked to the shell and extend from the outer surface.

The PBPs, FBPs, and optionally, FMPs, endothelial cell targeting peptides, and/or other platelet targeting peptides can be conjugated to the nanoparticle surface by reacting the N-termini of the peptides to the carboxyl termini of a heterobifunctional polyethylene glycol (PEG), such as maleimide-PEG-COOH. The PEG-peptide conjugates or PEGylated peptides can then be conjugated to the nanoparticle using known conjugation techniques. By way of example, a PBP having the amino acid sequence of SEQ ID NO: 1 (CGSSSGRGDSPA) can be conjugated to DSPE-PEG-Mal through thioether chemistry utilizing the thiol (—SH) group of the peptide cysteine residue. The FBP sequence having the amino acid sequence of SEQ ID NO: 2 (cyclo-AC-Y(DGI)C(HPr)YGLCYIQGK-Am) can be conjugated to DSPE-PEG-NHS through amide chemistry via the amine on the lysine.

In other embodiments, the PBPs, FBPs, and optionally, FMPs, endothelial cell targeting peptides, and/or other platelet targeting peptides can be conjugated to lipids that define the nanoparticle surface with PEG acrylate, PEG diacrylate, or other molecules of a variety of molecular weights.

The PEG molecules can have a variety of lengths and molecular weights, including, for example, PEG 200, PEG 1000, PEG 1500, PEG 2000, PEG 4600, PEG 10,000, or combinations thereof. In some embodiments, the PEG has molecular weight of 2000.

In some embodiments, the PBP and FBP conjugated phospholipids can include about 1 mole % to about 10 mole %, preferably about 2.5 mole % to about 10 mole % of the total lipid composition of the liposome.

In some embodiments, the CTNP liposome can include DSPE conjugated to PBP with PEG (DSPE-PEG-PBP), DSPE conjugated to FBP with PEG (DSPE-PEG-FBP), thrombin-cleavable lipopeptide conjugate, and cholesterol. The CTNPs can be formed by dissolving the phospholipids (e.g., DSPC and/or DHPE), clot-targeting peptide-lipid conjugates (e.g., DSPE-PEG-PBP and DSPE-PEG-FBP), thrombin-cleavable lipopeptide conjugate (e.g., Stearylamine-TCP) in a chloroform:methanol solution and using thin film evaporation to form a mixed lipid film. The lipid film can then be rehydrated with a plasmin solution in saline (e.g., about 425 g/mL concentration), and sonicated to form the CTNPs. Optionally, post sonication and lipid film resuspension, the resultant multilamellar vesicles can be extruded through membrane filters with, for example, about 200 nm-sized pores, using a liposome extruder to form unilamellar CTNP vesicles.

It will be appreciated that other therapeutic agents or bioactive agents can be encapsulated by, contained in, and/or linked to the CTNPs besides plasmin. Such therapeutic agents or bioactive agents can include any substance capable of exerting a biological or therapeutic effect in vitro and/or in vivo. In some embodiments, the therapeutic agent can be a thrombolytic agent that is encapsulated by, contained in, and/or linked to the CTNPs. Thrombolytic agents are used to dissolve blood clots in a procedure termed thrombolysis and can limit the damage caused by the blockage or occlusion of a blood vessel. Thrombolytic agents can include analogs of tissue plasminogen activator (tPA), the protein that normally activates plasmin and recombinant tissue plasminogen activators (r-tPAs). Examples of thrombolytic agents include alteplase, reteplase, and tenecteplase (sold under the trade name TNKase) and desmoteplase. Additional thrombolytic agents include anistreplase (sold under the trade name EMINASE), streptokinase (sold under the trade names KABIKINASE, STREPTASE), and urokinase (sold under the trade name ABBOKINASE).

In some embodiments, the therapeutic agent can be an anti-thrombotic agent that is encapsulated by, contained in, and/or linked to the CTNPs. Antithrombotic agents can include anticoagulants and antiplatelet agents.

Anticoagulants slow down clotting, thereby reducing fibrin formation and preventing clots from forming and growing. Anticoagulants include coumarins (vitamin K antagonists) such as coumadin. Anticoagulants also include but are not limited to heparin, heparin derivatives and direct thrombin inhibitors including the bivalent drugs hirudin, lepirudin, and bivalirudin and the monovalent drugs argatroban and dabigatran.

Antiplatelet agents prevent platelets from clumping and also prevent clots from forming and growing. Antiplatelet agents can include but are not limited to aspirin and clopidogrel (sold under the trade name PLAVIX).

In some embodiments, the additional therapeutic agents can be loaded into and/or onto the CTNPs by encapsulation, absorption, adsorption, and/or non-covalent linkage of the agent to or within the CTNPs. The amount of the additional therapeutic agent loaded onto or in the CTNPs can be controlled by changing the size of the CTNPs or the composition of the CTNPs.

In some embodiments, release of the additional therapeutic from the CTNPs can occur by desorption, diffusion through the polymer or lipid coating, or polymer or lipid wall, erosion, and/or disruption of the CTNP structure.

In some embodiments, the compositions comprising the CTNPs described herein, can be formulated in a pharmaceutical composition and administered to an animal, preferably a human, to facilitate the delivery of plasmin to a clot site. Formulation of pharmaceutical composition for use in the modes of administration noted below (and others) are described, for example, in Remington's Pharmaceutical Sciences (18^{th} edition), ed. A. Gennaro, 1990, Mack Publishing Company, Easton, Pa.

Such a pharmaceutical composition may consist of a plurality of the CTNPs alone, in a form suitable for administration to a subject, or the pharmaceutical composition may comprise a plurality of CTNPs and one or more pharmaceutically acceptable carriers, one or more additional ingredients, one or more pharmaceutically acceptable therapeutic agents, bioactive agents, imaging/diagnostic agents, or some combination of these. In some embodiments, the CTNPs and an additional therapeutic agent may be present in the pharmaceutical composition in the form of a physiologically acceptable ester or salt, such as in combination with a physiologically acceptable cation or anion, as is well known in the art. The pharmaceutically acceptable carrier can include a chemical composition with which the CTNPs and optional additional therapeutic agent may be combined and which, following the combination, can be used to administer the CTNPs and optional therapeutic agent to a subject.

For example, pharmaceutical compositions can be in the form of a sterile aqueous or oily injectable solution containing, if desired, additional ingredients, for example, preservatives, buffers, tonicity agents, antioxidants, stabilizers, nonionic wetting or clarifying agents, and viscosity increasing agents. This suspension or solution may be formulated according to the known art, and may comprise, in addition to the CTNPs, additional ingredients such as the dispersing agents, wetting agents, or suspending agents described herein. Such sterile injectable formulations may be prepared using a non-toxic parenterally acceptable diluent or solvent, such as water or 1,3 butane diol, for example. Other acceptable diluents and solvents include, but are not limited to, Ringer's solution, isotonic sodium chloride solution, and fixed oils such as synthetic mono or di-glycerides.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials.

In some embodiments, a composition including the CTNPs described herein can be used in a method for treating a vascular disease in a subject. In some embodiments, the disease can be characterized, in part, by the presence of activated platelets and/or platelet aggregation at a disease site (e.g., a thrombosed site). In some embodiments, a therapeutically effective amount of the composition can be administered in vivo to a subject to treat the subject. In some embodiments, an effective amount of the composition is the amount required to restore blood flow by about 50% from its thrombosed value.

It should be understood that the methods of treatment by the delivery of a composition including the CTNPs includes the treatment of subjects that are already afflicted with a vascular disease or symptoms thereof (e.g., a blood clot), as well as prophylactic treatment uses in subjects not yet afflicted and/or experiencing symptoms. In a preferred embodiment the subject is an animal. In a more preferred embodiment the subject is a human.

Although the descriptions of pharmaceutical compositions provided herein are principally directed to pharmaceutical compositions which are suitable for ethical administration to humans, it will be understood by the skilled artisan that such compositions are generally for administration to animals of all sorts. Modification of pharmaceutical compositions for administration to humans in order to render the compositions suitable for administration to various animals is well understood, and the ordinarily skilled veterinary pharmacologist can design and perform such modification with merely ordinary, if any, experimentation.

Pharmaceutical compositions that are useful in the methods described herein may be administered by any convenient route, such as by intravenous infusion or bolus injection. For example, the composition may be introduced into the subject by any suitable route, including intraventricular injection or intraventricular injection via an intraventricular catheter that is attached to a reservoir.

The composition can be delivered systematically (e.g., intravenously), regionally, or locally by, for example, intraarterial, intrathrombal, intravenous, parenteral, intraneural cavity, topical, oral or local administration, as well as subcutaneous, intra-tracheal (e.g., by aerosol), or transmucosal (e.g., buccal, bladder, vaginal, uterine, rectal, nasal, mucosal). If delivery of the composition to the brain is desired, the targeted composition can be injected into an artery of the carotid system of arteries (e.g., occipital artery, auricular artery, temporal artery, cerebral artery, maxillary artery etc.). As discussed above, the composition can be formulated as a pharmaceutical composition for in vivo administration.

The pharmaceutical compositions described herein may also be formulated so as to provide slow, prolonged or controlled release. In general, a controlled-release preparation is a pharmaceutical composition capable of releasing the CTNPs at a desired or required rate to maintain constant activity for a desired or required period of time.

The relative amounts of the CTNPs in a pharmaceutical composition of the invention will vary, depending upon the identity, size, and condition of the subject treated and further depending upon the route by which the composition is to be administered. By way of a non-limiting example, the composition may comprise between 0.1% and 100% (w/w) of the CTNPs.

The composition including the CTNPs can be administered to the subject at an amount effective to provide a desired result(s) and to avoid undesirable physiological results. In some embodiments, the CTNPs described herein may be administered to deliver a dose of between 1 ng/kg/day and 100 mg/kg/day. In one embodiment, a dose can be administered that results in a concentration of the CTNPs between 1 μM and 10 μM in a mammal. The precise dose to be employed can also depend on the route of administration, and should be decided according to the judgment of a medical practitioner and each subject's circumstances. In addition, known in vitro and in vivo assays may optionally be employed to help identify optimal dosage ranges. Effective doses may be extrapolated from dose-response curves derived from in vitro or in vivo test systems. Preferably, the dosage of the CTNPs will vary from about 1 g to about 50 mg per kilogram of body weight of the animal. More preferably, the dosage will vary from about 10 g to about 15 mg per kilogram of body weight of the animal. Even more preferably, the dosage will vary from about 100 μg to about 10 mg per kilogram of weight of the animal.

The composition can be administered in a variety of unit dosage forms, depending upon the particular disease or disorder being treated, the general medical condition of each subject, the method of administration, and the like. Details on dosages are well described in the scientific literature. The exact amount and concentration of the targeted compositions, or the “effective dose”, can be routinely determined (e.g., by a medical practitioner).

The pharmaceutical composition may be administered to a subject as needed. The pharmaceutical composition may be administered to a subject as frequently as several times daily, or it may be administered less frequently, such as once a day, once a week, once every two weeks, once a month, or even less frequently, such as once every several months or even once a year or less. The “dosing regimen” will depend upon a variety of factors, such as the type and severity of the disease being treated, the general state of the subject's health, the subject's age, and the like. Using guidelines describing alternative dosing regimens, e.g., from the use of other agents and compositions, the skilled artisan can readily determine by routine trials the optimal effective concentrations of the composition.

In many occlusive vascular diseases like stroke, myocardial infarction, peripheral arterial diseases and deep vein thrombosis, thrombo-occlusion and ischemia can deprive vital organs of circulation, oxygen and nutrients, leading to tissue and organ morbidity and often mortality. Rapid clot dissolution and revascularization is a mainstay in the clinical treatment of these critical disease conditions. Therefore, the application further relates to a method of treating a vascular disease in a subject. Vascular diseases and injuries treatable by the CTNPs described herein can include disease states or injuries characterized in part by the aggregation and/or adhesion of activated platelets in a disease site of subject. In some embodiments, the vascular disease or vascular injury includes an occlusive vascular disease such as but not limited to stroke, myocardial infarction, peripheral arterial diseases and deep vein thrombosis, thrombo-occlusion and ischemia.

In some embodiments, compositions described herein are administered immediately after it has been determined they are clinically appropriate. The advantage of administration is highest within the first sixty minutes after a thrombotic event, but may extend up to six hours after the start of symptoms. In some embodiments, the composition is administered in combination with anticoagulant drugs such as intravenous heparin or low molecular weight heparin, for synergistic antithrombotic effects and secondary prevention.

The following examples are for the purpose of illustration only and are not intended to limit the scope of the claims, which are appended hereto.

### Example

We developed a nanomedicine approach that can enable direct delivery of plasmin (instead of tPA) for rapid fibrinolytic effect. Direct systemic administration of plasmin has poor efficacy because it is rapidly (within <1 second) and irreversibly inhibited by inhibitors (e.g., antiplasmin) in circulation. Thus, the clinical standard of care has focused on intravenous administration of plasminogen activators (previously Streptokinase, and currently tPA that was approved in the United States in 1996), because such a plasminogen activator molecule can stay in circulation for longer time (e.g., tPA half-life in circulation is 5-10 min) and thereby can enable conversion of clot-associated plasminogen to plasmin for fibrinolytic action. However, tPA conversion of plasminogen to plasmin is not restricted to clot site only, but can occur in circulation on fibrinogen-bound plasminogen resulting in systemic fibrinogenolysis, and this has been implicated as a major cause of bleeding risks associated with tPA therapy.

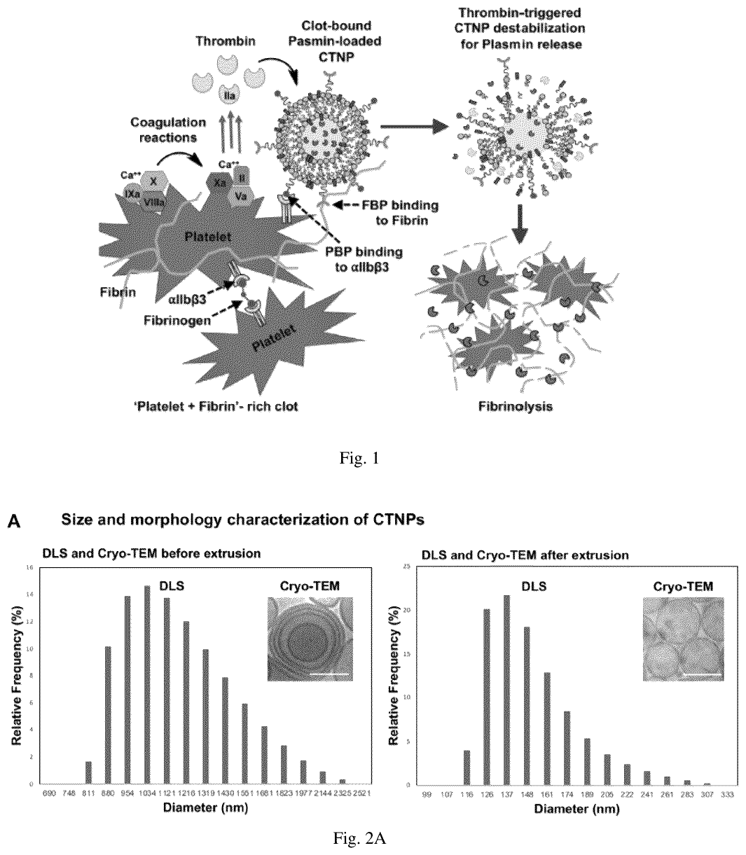

Compared with tPA, plasmin has an improved safety profile because its rapid neutralization by antiplasmin in circulation minimizes off-target risks, but this rapid neutralization is also the reason why plasmin cannot be used as a direct intravenous fibrinolytic therapy. Considering these challenges and opportunities, we developed a liposome-based nanomedicine system that: (i) can be systemically administered, whereas allowing specific anchorage onto clots via heteromultivalent binding of activated platelets and fibrin; (ii) undergo thrombin-triggered degradation of the lipid membrane to enable clot site-responsive particle destabilization for payload release; and (iii) encapsulate plasmin to protect it from neutralization in circulation, whereas enabling its release specifically at the clot site for localized fibrinolytic action. FIG. 1 depicts the design concept and mechanism of action for these plasmin-loaded heteromultivalently clot-targeted thrombin-cleavable nanoparticles (CTNPs).

### Materials and Methods

### Materials

For liposomal nanoparticle fabrication, distearoyl phosphotidyl choline (DSPC), methoxy polyethylene glycol-conjugated distearoyl phosphotidyl ethanolamine (DSPE-mPEG₂₀₀₀), and maleimide-terminated polyethylene glycol-conjugated distearoyl phosphotidyl ethanolamine (DSPE-PEG₂₀₀₀-Mal) were purchased from Avanti Lipids (Alabaster, USA). N-succinimide-terminated polyethylene glycol-conjugated distearoyl phosphotidyl ethanolamine (DSPE-PEG₂₀₀₀-NHS) was purchased from Nanosoft Polymers (Winston-Salem, USA). Platelet-binding peptide (PBP) sequence CGSSSGRGDSPA (SEQ ID NO: 1) that binds to activated platelet surface integrin αIIbβ3, fibrin-binding peptide (FBP) sequence cyclo-AC-Y(DGI)C(HPr)YGLCYIQGK-Am (SEQ ID NO: 2), and thrombin cleavable peptide (TCP) sequence DVTPRC (SEQ ID NO: 3) were custom synthesized by Genscript (Piscataway, USA). Polycarbonate membrane filters with 200 nm pore distribution, calcium chloride, stearylamine, diethyl ether, dimethyl sulfoxide, Sephadex G-100, and cholesterol were purchased from Sigma Aldrich (St. Louis, USA). Rhodamine-B-dihex-adecanoyl-sn-glycero-3-phosphoethanolamine (DHPE-RhB, red fluorescence, λₑₓ=561, λₑₘ=582) was purchased from Setareh Biotech (Eugene, USA). Thrombin, plasmin, and α2-antiplasmin were purchased from Hematological Technologies (Essex Junction, USA). Phosphate buffered saline, tris(hydroxymethyl)aminomethane hydrochloride (Tris-HCl), succinimidyl 3-(2-pyridyldithio)propionate (SPDP), fluted filter paper, collagen type I from rat tail, Chrono Log Corporation collagen type I, and AlexaFluor 647-conjugated fibrinogen were purchased from Fisher Scientific (Waltham, USA). Calcein AM was purchased from Thermo Fisher (Waltham, USA). Liposome extruder for nanoparticle manufacture was purchased from Evonik (Essen, Germany). For microfluidic studies, the parallel plate flow chamber (PPFC) was purchased from Glycotech (Gaitersberg, USA), and BioFlux flow controller and microfluidic plates were purchased from Fluxion Biosciences (Alameda, USA). All human blood and plasma for in vitro studies were obtained either from healthy donors using protocol approved by Case Western Institutional Review Board (Case IRB STUDY20191092) or from the Case Western He-matopoietic Biorepository and Cellular Therapy Shared Resource core facility that provides de-identified human blood for research purposes.

### Manufacture of CTNPs

The CTNPs are manufactured utilizing the liposomal self-assembly of several different lipid molecules. For clot-targeting capability, a combination targeting activated platelets and fibrin was used because these are the major components of a thrombus. In fact, we have previously demonstrated that such combination targeting utilizing heteromultivalent decoration of ligand motifs on nanoparticles can enhance the targeting specificity and efficacy under a hemodynamic flow environment. For targeting active platelets, the PBP sequence CGSSSGRGDSPA (SEQ ID NO: 1) was conjugated to DSPE-PEG₂₀₀₀-Mal through thioether chemistry utilizing the thiol (—SH) group of the peptide cysteine residue. For targeting to fibrin, the FBP sequence cyclo-AC-Y(DGI)C(HPr)YGLCYIQGK-Am (SEQ ID NO: 2) was conjugated to DSPE-PEG₂₀₀₀-NHS through amide chemistry via the amine on the lysine. The conjugation products were confirmed using matrix-assisted laser desorption ionization time-of-flight (MALDI-TOF) mass spectroscopy. For rendering the thrombin-responsive degradation property of the nanoparticles, a thrombin-cleavable lipopeptide conjugate was formed via two-step reaction. First, stearylamine was conjugated to succinimidyl 3-(2-pyridyldithio)propionate (SPDP) in dimethyl sulfoxide via amide chemistry utilizing the amine group on stearylamine and the NHS ester on the SPDP. Subsequently, the TCP DVTPRC (SEQ ID NO: 3) was conjugated to the pyridyl end of SPDP via disulfide chemistry using the thiol (—SH) group on the TCP. Excess peptide was removed by vacuum filtration and washing the conjugate with deionized water, and the resultant conjugate was purified by liquid-liquid extraction with deionized water and ethyl ether. The conjugate was characterized by MALDI-TOF mass spectroscopy and lyophilized until further use. The thrombin-cleavable property of the conjugate was characterized by exposing the conjugate to 250 nM thrombin in saline at 37° C. for 30 minutes and confirming degradation of the conjugate by MALDI-TOF mass spectroscopy. Following confirmation of all lipid-peptide conjugates, DSPC (44 mol %), DHPE-RhB (1 mol %), cholesterol (20 mol %), thrombin-cleavable lipopeptide conjugate (Stearylamine-TCP, 30 mol %), and clot-targeting peptide-lipid conjugates (DSPE-PEG2000-PBP and DSPE-PEG2000-FBP, 2.5 mol % each), were dissolved in 1:1 chloroform:methanol, and a mixed lipid film was formed using thin film evaporation. The lipid film was rehydrated with saline (for targeting studies only) or a plasmin solution in saline (425 g/mL concentration, for fibrinolysis studies), and was sonicated for 1 hour to form CTNPs. Post sonication and lipid film resuspension, the resultant multilamellar vesicles were extruded through polycarbonate membrane filters with 200 nm-sized pores using a liposome extruder to form unilamellar CTNP vesicles. Control (undecorated) nanoparticles were manufactured similarly, but instead of using the DSPE-PEG₂₀₀₀-peptide components, DSPE-PEG-mPEG₂₀₀₀ was used. CTNPs as well as the control nanoparticles were characterized for size using dynamic light scattering (DLS) and cryo-transmission electron microscopy (cryo-TEM). For plasmin-loaded CTNPs used in fibrinolysis studies, unencapsulated plasmin was separated from the nanoparticles using Sephadex G-100 bead columns. Plasmin-loaded CTNPs were incubated with Triton X-100 to assess “exhaustive release” (100% release because of complete particle disassembly). For characterizing plasmin release from CTNPs via diffusion only (without thrombin trigger) as well as via “diffusion+particle destabilization” (with thrombin trigger), CTNPs (3×10¹¹ nanoparticles per mL) were incubated in 2 mL Eppendorf tubes for 2 hours at 37° C., in absence or in presence of thrombin (250 nM) and antiplasmin (1 μM). For both conditions, sample aliquots (100 μL) were taken at various time intervals over 2 hours and analyzed by plasmin-antiplasmin (PAP) ELISA to calculate plasmin released. All data pertaining to diffusion-mediated release and thrombin-triggered particle destabilization-mediated release were normalized to the “exhaustive release” data.

### Microfluidics-Based Evaluation of Clot Targeting by CTNPs

Platelet-rich plasma (PRP) with calcein-stained platelets and 5% v/v AlexaFluor-647 fibrinogen was mixed with 250 nM thrombin in 0.5 M CaCl₂) was incubated on collagen-coated glass slides to form “platelet+fibrin”-rich clots showing blue platelets and green fibrin. The clot-bearing slides were sealed into a GlycoTech PPFC and was washed with saline for 5 minutes. The clot was exposed to Rhodamine B (RhB)-labeled empty CTNPs (peptide-decorated but no plasmin encapsulation) or control nanoparticles (no peptide decoration), in saline under a shear stress of 25 dyn/cm², and were allowed to flow in a closed loop over the clot for 30 minutes. The clot was washed with saline for 15 minutes to remove unbound particles and imaged under inverted epifluorescence microscope. With the imaged area maintained constant for all studies, surface-averaged RhB (red) fluorescence intensity was recorded for control nanoparticle vs. CTNPs bound to the clot, and analyzed statistically to quantify nanoparticle binding.

Fibrinolysis with Plasmin-Loaded CTNPs In Vitro

The fibrinolytic efficacy of plasmin-loaded CTNPs was first evaluated in vitro in a well-plate assay. For this, clots were formed in well plates by incubating PRP (50 μL) with 250 nM thrombin in calcium for 2 hours. The clots were washed with saline to remove residual plasma, and then incubated with free plasmin, plasmin-loaded CTNPs without thrombin or plasmin-loaded CTNPs with thrombin (250 nM), or Tris-HCl buffer (pH 8) for 30 minutes at 37° C. Sample aliquots (100 μL) were taken at various time intervals (5, 15, and 30 minutes time points), and D-dimer ELISA was performed on the samples to determine the amount of clot degradation. For each treatment group, 3 replicates of this experiment were used for analysis. The targeted fibrinolytic efficacy of the plasmin-loaded CTNPs was then evaluated under flow using the Bioflux microfluidic device. Microfluidic channels were coated with equine collagen type IV and von Willabrand Factor (vWF) by incubating for 1 hour. The channels were washed with saline, and PRP with calcein AM-stained platelets and AlexaFluor-647 fibrinogen was flowed in the channel for 10 minutes at 60 dyn/cm² to form a fibrin-rich clot. The clot was washed with saline, and plasmin-loaded CTNPs with or without clot-targeting capabilities were flowed over the clot for 30 minutes at 25 dyn/cm². Free plasmin in saline as well as plasmin introduced with antiplasmin were used as positive and negative controls, respectively. The clot was imaged using an inverted fluorescent microscope over 0-30 minutes, the images were analyzed for surface-averaged fibrin (green) fluorescence intensity in the fixed channel area, and clot degradation kinetics was measured as a decrease in this fibrin fluorescence intensity over 30 minutes normalized to the fibrin fluorescence at t=0 min. Additionally, at the 30 minutes time-point for each experiment, the fluid in the outlet well was collected and analyzed by D-dimer ELISA to further quantify fibrin degradation. The studies were run in triplicate for statistical data analysis.

Fibrinolysis with Plasmin-Loaded CTNPs In Vivo in a Zebrafish Thrombosis Model

Zebrafish (Danio rerio) were raised in accordance with animal care guidelines as approved by the University of Michigan Animal Care and Use Committee. Fish strains were acquired from the Zebrafish International Resource Center and all experiments were performed in an AB×TL hybrid. Laser-mediated endothelial injury was used to produce occlusive thrombosis at 3 days post-fertilization (3 dpf) in the zebrafish posterior cardinal vein (PCV). Larvae were first anesthetized in tricaine and mounted in 0.8% low melting point agarose on glass coverslips. The agarose around the head of the larvae was removed and 3 nl of CTNPs or 1.75 nl free plasmin (72.9 g/mL) were infused retro-orbitally via the anterior cardinal vein. For time-to-occlusion (TTO) studies, this treatment administration was performed first, followed by laser injury of the PCV endothelium 5 somites caudal to the anal pore using 99 pulses at power level 18 (MicroPoint Pulsed Laser System, Andor Technology). TTO of the PCV was observed up to 2 minutes and then larvae were checked for successful infusion of CTNPs by the presence of fluorescence in circulation in all assays except for free plasmin. For time-to-recanalization (TTR) studies, venous laser injury was performed first, followed by infusion of CTNPs into the anterior cardinal vein, and TTR observed up to 30 minutes. For control conditions, TTO and TTR experiments were performed without or with empty nanoparticles, as well as with plasmin-loaded clot-targeted nanoparticles (CNPs) that do not have the thrombin-cleavable component.

### Statistical Analysis

For in vitro clot-binding studies of nanoparticles, paired t-test was performed for statistical analysis based on quantification of nano-particle RhB intensity bound to the clot, and significance was considered for p<0.05. For well plate-based fibrinolysis assays, one-way analysis of variance tests were used for the statistical analysis, and significance was considered for p<0.05. Comparisons were carried out between free plasmin versus plasmin-loaded CTNPs with or without thrombin versus Tris-HCl buffer. For BioFlux microfluidic-based fibrinolysis studies and associated D-dimer ELISA, one-way analysis of variance tests were used for the statistical analysis, and significance was considered for p<0.05. Comparisons were carried out between free plasmin versus plasmin-loaded CTNPs with or without thrombin versus free plasmin incubated with antiplasmin. For zebra-fish thrombosis TTO and TTR studies, statistical comparison of treatment groups were performed using the Mann-Whitney U test.

### Results

### Manufacture and Characterization of CTNPs

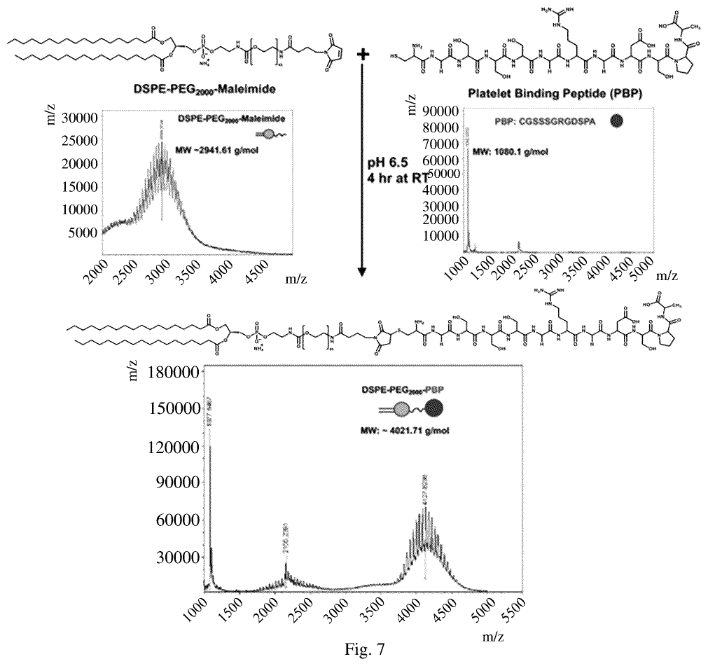

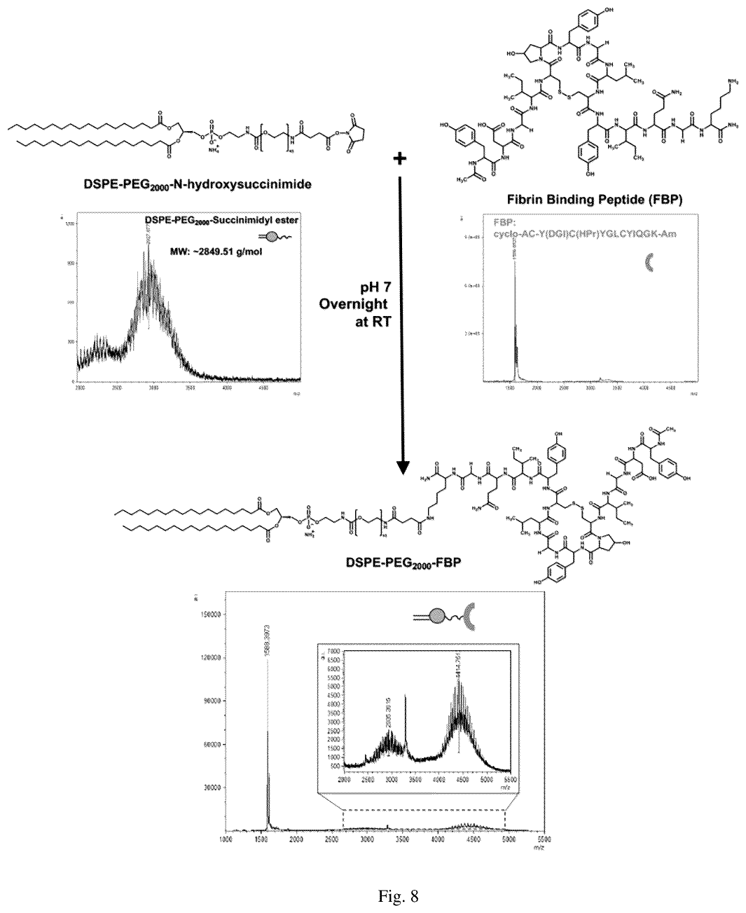

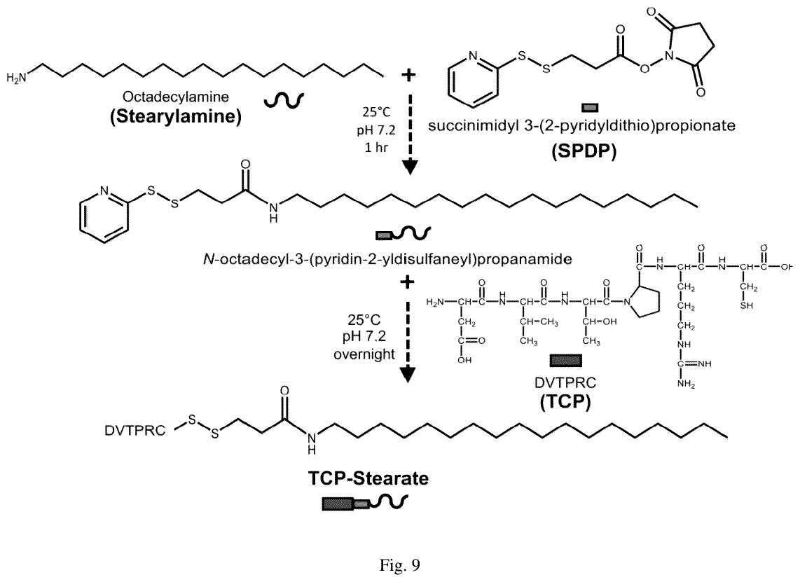

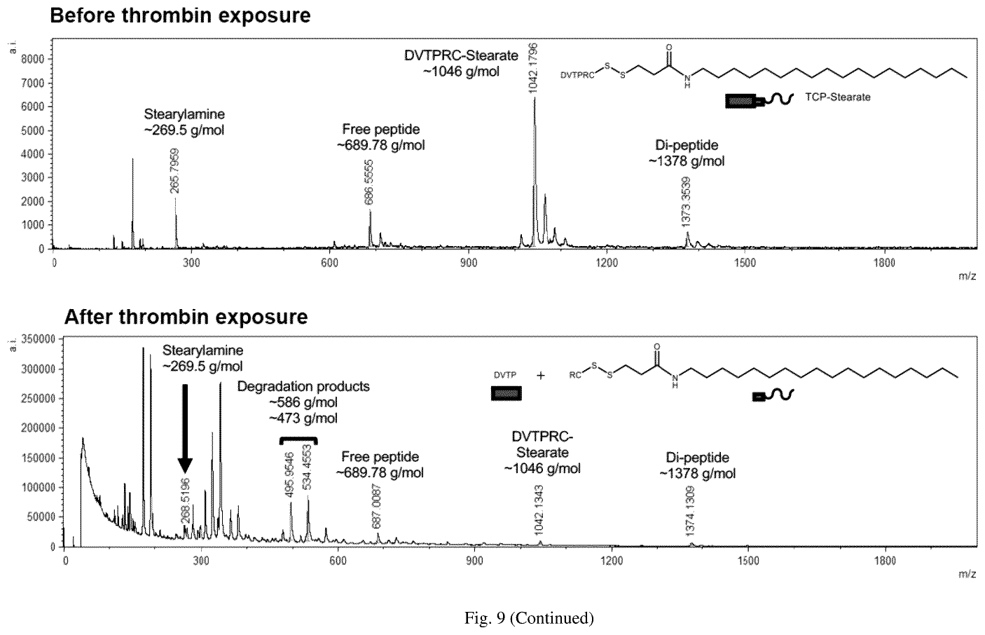

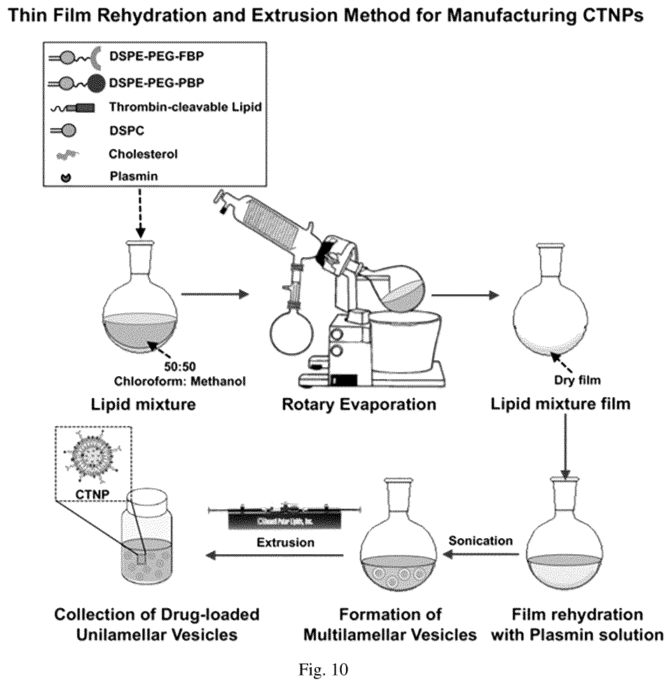

FIGS. 7 and 8 show the peptide-lipid conjugation schematic as well as representative MALDI-TOF mass spectroscopy data for the DSPE-PEG₂₀₀₀-PBP and DSPE-PEG₂₀₀₀-FBP synthesis. FIG. 9 shows the reaction schematic for the synthesis of the thrombin-cleavable lipopeptide conjugate (TCP-stearate), as well as representative MALDI-TOF mass spectroscopy characterization of the TCP-stearate molecule before and after exposure to thrombin (30 minutes, 250 nM) to confirm thrombin-induced cleavage. The results demonstrate that thrombin could efficiently cleave the conjugate, evidenced by the fact that after thrombin exposure very little of the TCP-stearate conjugate remains (peak at 1046 m/z), whereas 2 new degradation peaks appear (495 and 534 m/z) indicative of the degradation products. FIG. 10 shows the schematic for plasmin-loaded CTNP manufacture using thin film rehydration and extrusion method, resulting in nanoparticles ˜150 to 200 nm in diameter.

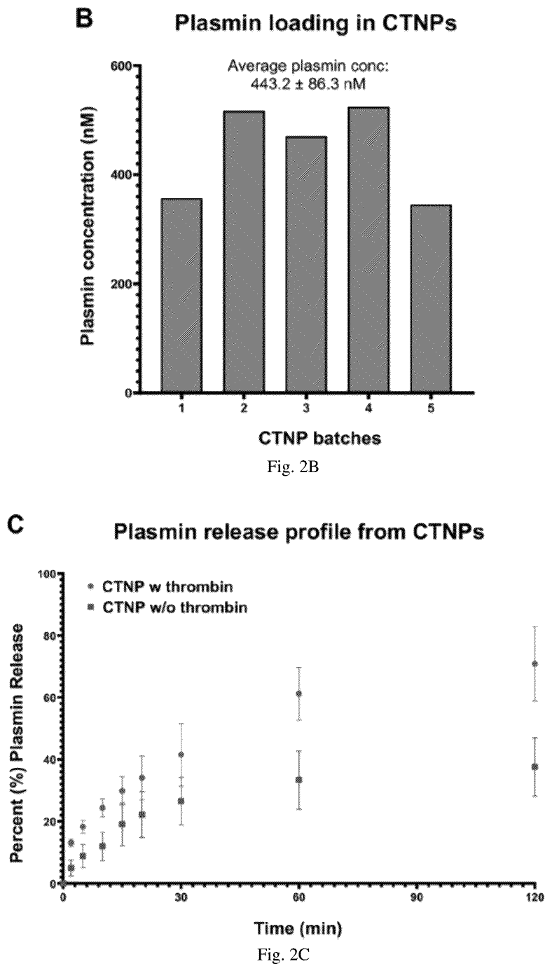

FIG. 2A shows representative DLS characterization data and cryo-TEM images of the vesicles before extrusion and after extrusion, indicating that after extrusion the unilamellar vesicles had a size of ˜150 to 200 nm diameter. FIG. 2B shows plasmin loading results across 5 representative CTNP batches, indicating that per batch (3×10¹² nanoparticles per mL) the encapsulated plasmin concentration is 443.2±86.3 nM. FIG. 2C shows release kinetics of plasmin from such CTNPs, indicating that without exposure to thrombin the diffusive release of plasmin is low (˜40% over a 2-hour period), whereas upon exposure to thrombin the release is significantly increased to ˜70% during the same period. This enhanced release of plasmin upon exposure of CTNPs to thrombin can be attributed to thrombin-induced cleavage of the TCP-stearate in the CTNP shell, which destabilizes the particles.

### Clot-Targeting Capability of CTNPs In Vitro

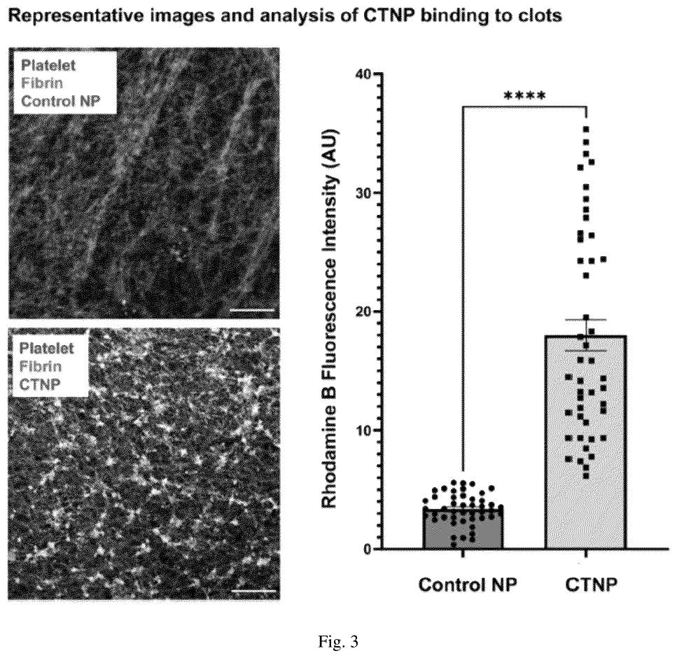

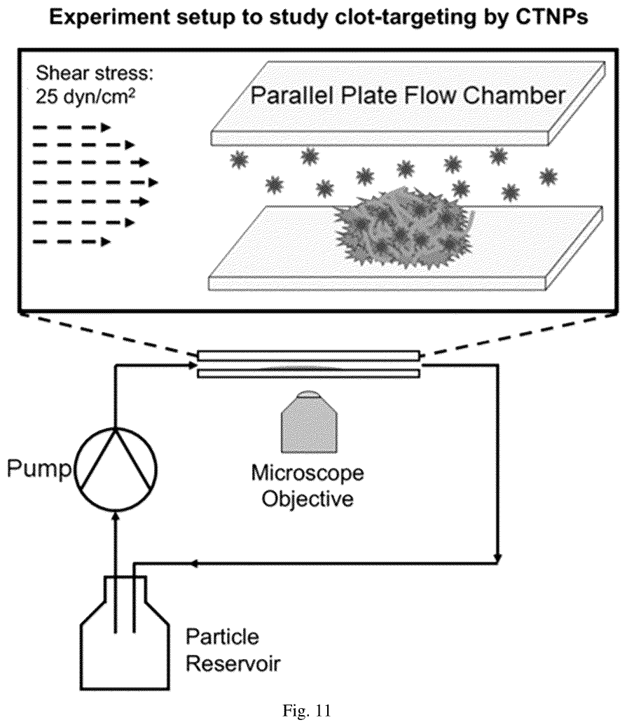

FIG. 11 depicts the PPFC microfluidic setup for evaluating the clot-targeting capability of CTNPs under a vascularly relevant shear flow (25 dyn/cm²) environment. FIG. 3 shows representative confocal fluorescence images at the experiment end-point (30 min), as well as analyzed quantitative data for surface-averaged RhB fluorescence intensity from these targeting studies. As evident from FIG. 3, control nanoparticles showed minimal binding to the clots, whereas CTNPs showed substantial binding, co-localizing with both platelets (appearing purple in the image) and fibrin (appearing yellow in the image). Quantitative analysis of the RhB fluorescence intensity clearly indicated that the CTNPs had significantly higher (p≤0.0001) binding to clots, than control nanoparticles. The nonzero binding of control nanoparticles to clots is possibly a result of nonspecific binding as some lipidic nanoparticles can fuse with the platelet cell membrane as well as get physically trapped in the clot mesh. These results indicate that the combination of platelet-binding (via PBP) and fibrin-binding (via FBP) mechanisms renders clot-specific high binding ability of the CTNPs.

Fibrinolysis with Plasmin-Loaded CTNPs In Vitro

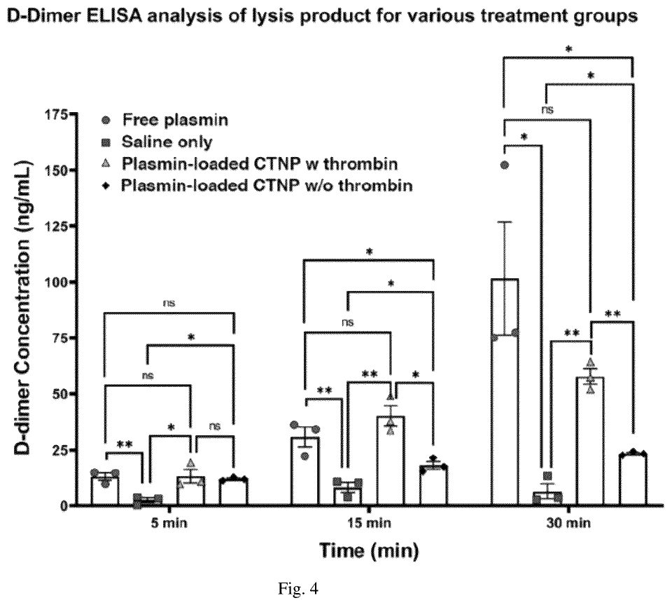

FIG. 4 shows quantitative data from the D-dimer analysis of the well-plate-based fibrinolysis studies where “platelet+fibrin”-rich clots in wells were incubated for 1-hour with free plasmin (positive control), or saline only (negative control), or plasmin-loaded CTNPs with versus without exogenously added thrombin (to simulate the thrombin-rich environment of clots). Elevated D-dimer is a marker of increased fibrinolysis. The results indicate that over the 30-minute period free plasmin could significantly degrade the fibrin clot (increasing D-dimer level), whereas saline was unable to cause any substantial clot degradation (low D-dimer value). The plasmin-loaded CTNPs rendered only modest degradation of clots when thrombin was not added exogenously to the wells, but in the presence of thrombin the plasmin-loaded CTNPs rendered significantly higher fibrinolysis. This suggests that in absence of thrombin the small amount of diffusively released plasmin from CTNPs can cause a modest extent of fibrinolysis, but the fibrinolytic effect is significantly enhanced when plasmin release from CTNPs is increased by thrombin trigger.

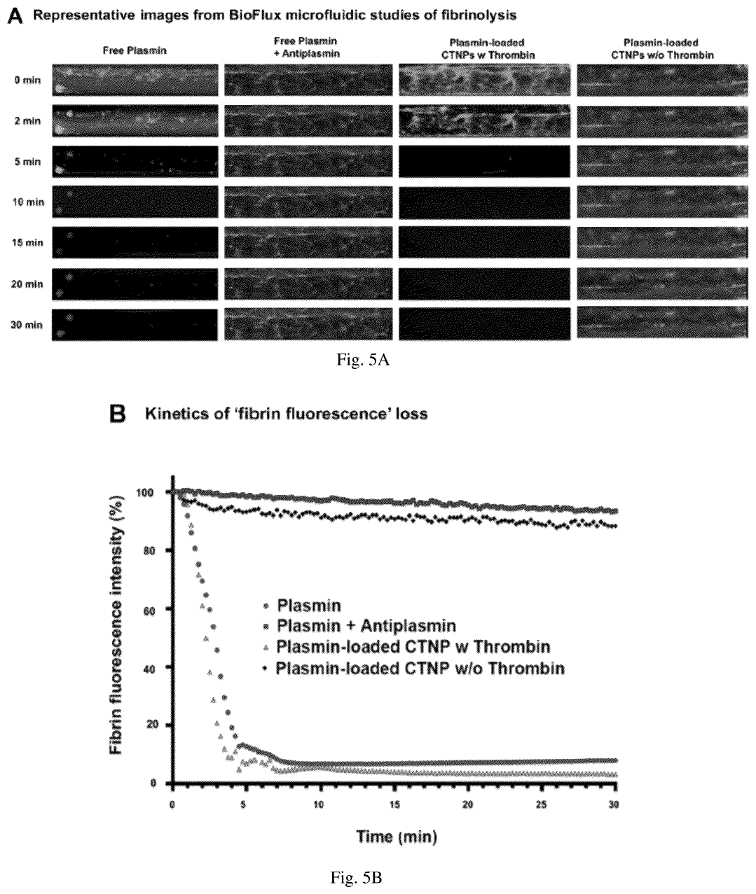

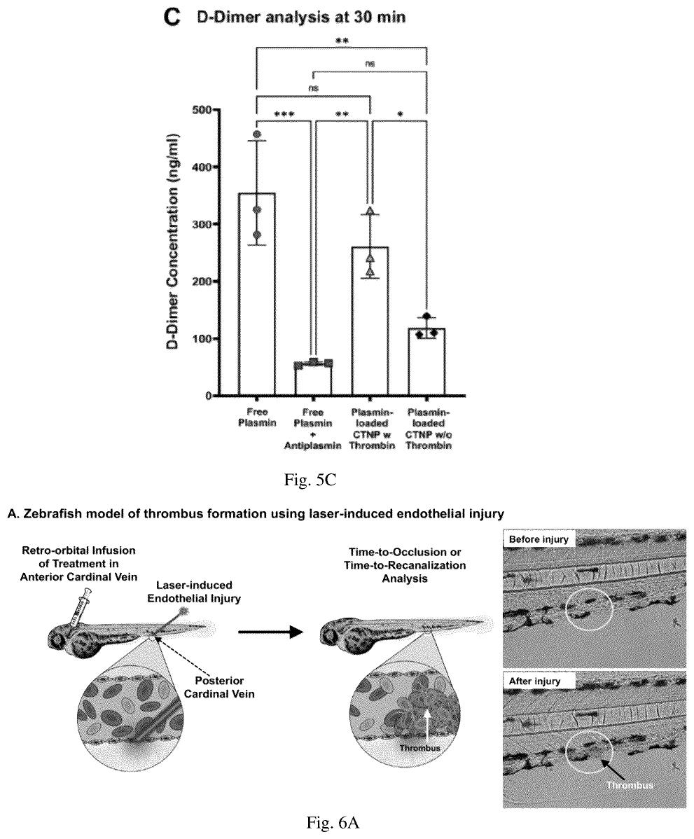

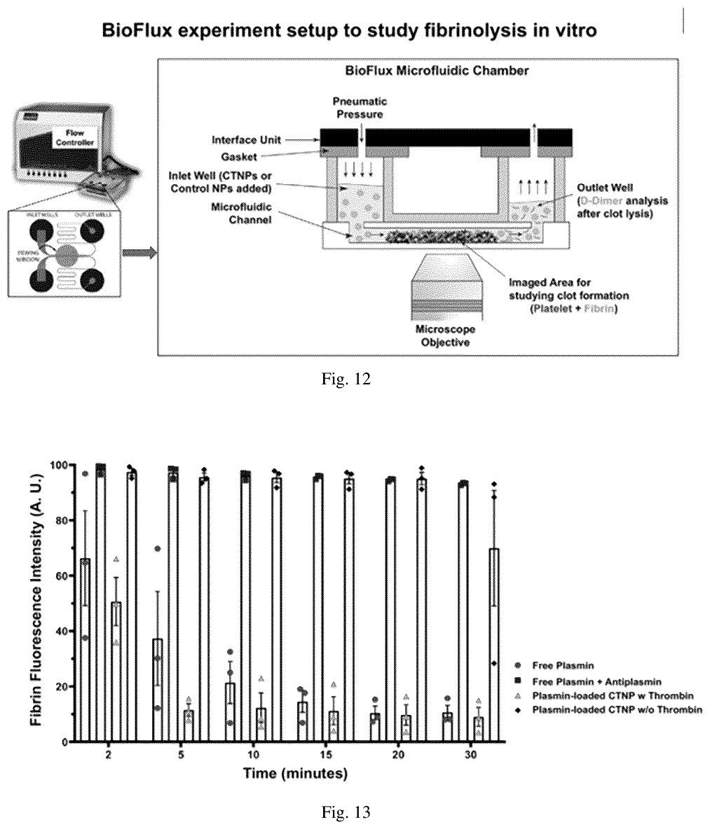

Building on the above-described demonstration of plasmin-loaded CTNPs to render increased fibrinolysis in presence of thrombin, subsequent studies were performed to evaluate whether this capability of CTNPs is conserved under a simulated vascular flow environment. For this, a BioFlux microfluidic system was used (schematically shown in FIG. 12) where PRP clots (blue platelets, green fibrin) were formed by 2-hour incubation in “collagen+vWF”-coated microchannels, washed with saline to remove residual liquid, and then exposed to flow of free plasmin (positive control), or “free plasmin+antiplasmin” added together (negative control), or plasmin-loaded CTNPs without versus with exogenously added thrombin (to simulate a thrombin-rich clot environment). Clot lysis was imaged for 30 minutes, and the loss of fibrin fluorescence (green) over time was assessed as an indicator of fibrinolysis. FIG. 5A shows representative images from these studies under various treatment groups over the 30-min period, and FIG. 5B shows kinetic analysis of “fibrin fluorescence loss” over the 30-min period in the imaged microfluidic area. FIG. 13 shows “fibrin fluorescence intensity” data from image analysis in the channel area in these experiments at various timepoints over the 30-minute period.

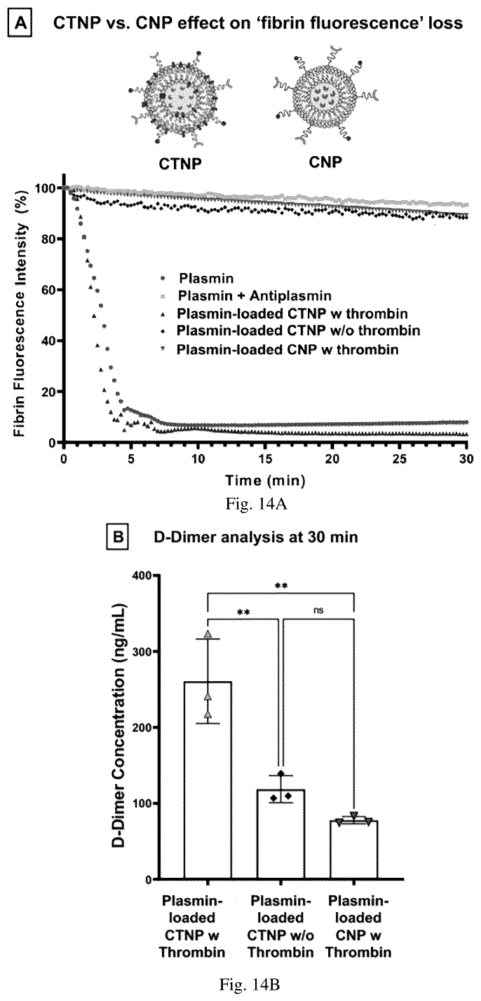

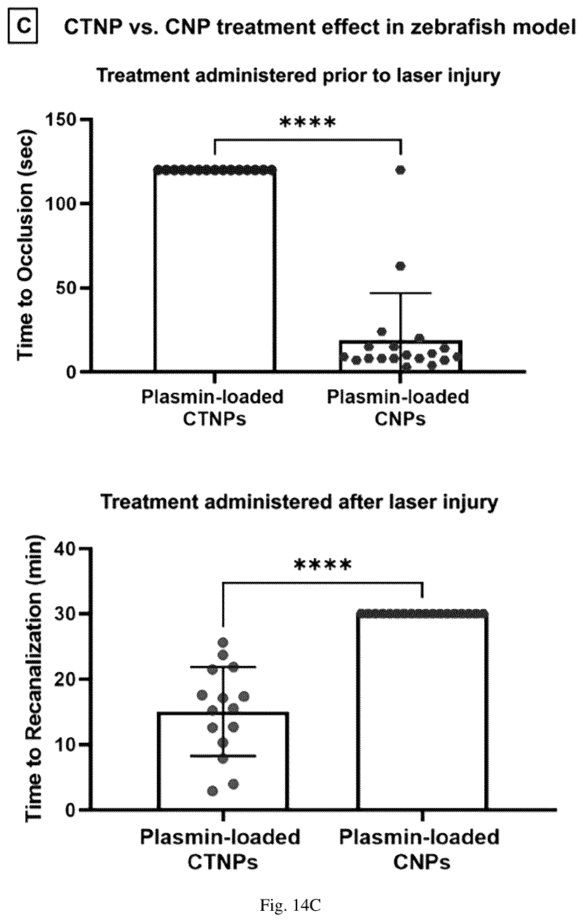

As evident from these results, free plasmin was able to rapidly lyse the clot (sharp drop in fibrin fluorescence), and this effect was majorly inhibited when antiplasmin was added to the system. Plasmin-loaded CTNPs without thrombin exposure caused minimal fibrinolysis, whereas on exposure to thrombin these particles were able to render substantial clot lysis, comparable to the effect of free plasmin. As an additional comparison, plasmin-loaded clot-targeted nanoparticles without the thrombin-cleavable lipid component in the particle shell were prepared (termed clot-targeted nanoparticle or CNP) and flowed over clots in presence of exogenously added thrombin, in the BioFlux channels. Thus, “plasmin-loaded CTNPs without thrombin exposure” and “plasmin-loaded CNPs with thrombin exposure” were the 2 complementary groups to assess whether the thrombin-triggered plasmin release property is necessary for enhanced fibrinolysis. The comparison data are shown in FIG. 14A where the “fibrin fluorescence loss” from treatment of the clots with plasmin-loaded CTNPs without thrombin or plasmin-loaded CNPs with thrombin were both minimal compared with treatment with plasmin-loaded CTNPs plus thrombin. These results strongly indicate that the thrombin-triggered enhanced release of plasmin from CTNPs can render significantly higher fibrinolysis compared with when such triggered release is not present and only diffusive release is present. Additional analyses were done by collecting the lysis products from the outlet well at the 30-minutes time-point for the various treatment groups and analyzing D-dimer concentration, as shown in FIG. 5C. These results further indicate that plasmin-loaded CTNPs in the absence of thrombin exposure resulted in low fibrinolysis (low D-dimer values), whereas in the presence of thrombin these CTNPs rendered significantly higher fibrinolysis. FIG. 14B shows additional comparisons from D-dimer analysis of the lysis products collected from these experiments, further confirming that the low amount of plasmin diffusively released from CTNPs in the absence of thrombin or from CNPs in the presence of thrombin is unable to cause substantial lysis under a flow environment, whereas the enhanced release of plasmin from CTNPs in the presence of thrombin can increase fibrinolysis.

Fibrinolysis with Plasmin-Loaded CTNPs In Vivo in the Zebrafish Model

FIG. 6A shows the general setup for the zebrafish studies where laser-induced endothelial injury in the PCV was used to create occlusive venous thrombi. It is well-reported that such laser-induced venous thrombosis in zebrafish is fibrin-rich, and thus, it was considered an appropriate model to study fibrinolytic effect of plasmin-loaded CTNPs. For TTO studies, the treatments were administered before laser injury, with the rationale that the fibrinolytic effect of the treatment, if any, would prevent (or delay) vessel thrombo-occlusion. For TTR studies, the treatments were administered after endothelial injury and clot formation, testing the ability of the treatment to render fibrinolysis and subsequent recanalization. FIG. 6B shows results from TTO studies and FIG. 6C shows results from TTR studies in the zebrafish thrombosis model. As evident from FIG. 6B, larvae without treatment or with “control nanoparticle treatment” exhibited rapid vessel occlusion (in <25 seconds). Interestingly, this was also observed for “free plasmin treatment” group, likely because the plasmin is rapidly inhibited on administration thereby preventing any therapeutic action. In contrast, treatment with plasmin-loaded CTNPs were able to significantly prevent clot formation such that there was no vessel occlusion observed for 120 sec (final time-point of experiment).

These results indicate that encapsulating the plasmin within CTNPs protected it from rapid inhibition, and plasmin was released from CTNPs as the particles started binding to the clot site and undergoing thrombin-triggered destabilization. As evident from FIG. 6C, larvae without treatment or with “control nanoparticles” showed no sign of recanalization within 30 min, whereas larvae with plasmin-loaded CTNPs recanalized in <20 minutes. This suggests that plasmin released from clot-anchored CTNPs was able to render effective fibrinolysis to recanalize the vessel. Additional TTO and TTR study results comparing plasmin-loaded CTNPs (thrombin-triggered mechanism present) versus plasmin-loaded CNPs (thrombin-triggered mechanism absent) in the zebra-fish model are shown in FIGS. 14C and 14D. These data confirm that the thrombin-triggered enhanced plasmin release mechanism is necessary to achieve fibrinolysis, compared with when low amounts of plasmin are released only by diffusion.

From the above description of the invention, those skilled in the art will perceive improvements, changes and modifications. Such improvements, changes and modifications within the skill of the art are intended to be covered by the appended claims. All references, publications, and patents cited in the present application are herein incorporated by reference in their entirety.

Having described the invention, the following is claimed:

1. A composition comprising:
a plurality of clot-targeted thrombin cleavable nanoparticles (CTNPs), each CTNP including a thrombin cleavable shell that defines an outer surface of the CTNP, a core, which is loaded with plasmin, and a plurality of platelet binding peptides (PBPs) and fibrin binding peptides (FBPs) that are linked to the shell and extend from the outer surface, wherein the CTNP is configured to adhere to clots, activated platelets, and/or fibrin upon systemic administration to a subject, shield the loaded plasmin in circulation from neutralization, and release the loaded plasmin at the clot site by thrombin triggered degradation of the CTNP.

2. The composition of claim 1, wherein the CTNPs bind to the clot under a hemodynamic shear environment.

3. The composition of claim 1, wherein the CTNPs provide localized thrombolytic and/or fibrinolytic action at the clot site.

4. The composition of claim, wherein the shell of the CTNP includes at least one phospholipid and a thrombin cleavable lipopeptide conjugate that triggers degradation of the CTNP and release of the plasmin at the clot site.

5. The composition of claim 1, wherein the CTNP has a diameter of about 50 nm to about 5 km.

6. The composition of claim 1, wherein the CTNP is a liposome.

7. The composition of claim 6, wherein the liposome includes a plurality of phospholipids and optionally cholesterol to define a lipid membrane.

8. The composition of claim 7, wherein the phospholipids include at least one of distearoylphosphatidylserine (DSPS), distearoylphosphatidylcholine dipalmitoylphosphatidylcholine (DSPC), dihexadecanoylglycerophosphoethanolamine (DHPE), dibehenoylglycerophosphocoline (DBPC), distearoylphosphatidylcholine (DSPC), diarachidonylphosphatidylcholine (DAPC), dioleoylphosphatidylethanolamine (DOPE), dipalmitoylphosphatidylethanolamine (DPPE), and distearoylphosphatidylethanolamine (DSPE); dipalmitoylphosphatidic acid (DPPA), or PEG functionalized lipids thereof.

9. The composition of claim 7, wherein the PBPs and FBPs are conjugated to the phospholipids with PEG linkers.

10. The composition of claim 9, wherein the PBP and FBP conjugated phospholipids comprise about 1 mole % to about 10 mole % of the total lipid composition of the liposome.

11. The composition of claim 6, wherein the liposome comprises DSPE conjugated to PBP with PEG (DSPE-PEG-PBP), DSPE conjugated to FBP with PEG (DSPE-PEG-FBP), thrombin-cleavable lipopeptide conjugate, and cholesterol.

12. The composition of claim 1, wherein the PBPs and FBPs are spatially or topographically arranged on the outer surface such that the PBPs and FBPs do not spatially mask each other and the nanoparticle is able to adhere to a clot site with exposed activated platelet surface integrin αIIβ3 and fibrin.

13. The composition of claim 1, wherein the CTNPs have a shape, size and elastic modulus that facilitates margination to a clot site upon administration to vasculature of a subject.

14. The composition of claim 1, wherein the PBPs have an amino acid sequence of SEQ ID NO: 1 (CGSSSGRGDSPA) and the CBPs have an amino acid sequence of SEQ ID NO: 2 (cyclo-AC-Y(DGI)C(HPr)YGLCYIQGK-Am).

15. The composition of claim 1, wherein the ratio of PPB:FPB: is about 25:75 to about 75:25.

16. The composition of claim 1, wherein the thrombin cleavable lipopeptide conjugate includes a thrombin cleavable peptide.

17. The composition of claim 16, wherein the thrombin cleavable peptide has an amino acid sequence of SEQ ID NO: 3(DVTPRC).

18. The composition of claim 1, wherein the amount of plasmin delivered to a clot site in a subject by the CTNPs is an amount effective to promote thrombolysis and/or fibrinolysis of the clot in the subject.

19. A method of delivering plasmin to a clot site in a subject, the method comprising administering to the subject a composition of claim 1.

20. A method of treating a clot in vasculature of a subject in need thereof, the method comprising administering to the subject a composition of claim 1.


SEQUENCE LISTING (3 sequences)

SEQ ID NO: 1 - protein, 12 aa, synthetic construct
CGSSSGRGDSPA

SEQ ID NO: 2 - protein, 15 aa, synthetic construct
YDGICPYGLCYIQGK

SEQ ID NO: 3 - protein, 6 aa, synthetic construct
DVTPRC